(19) United States
(12) Patent Application Publication
(10) Pub. No.: US 2023/0407336 A1
(43) Pub. Date: Dec. 21, 2023

(54) HIGH-EFFICIENCY QUANTITATIVE CONTROL OF MITOCHONDRIAL TRANSFER BASED ON DROPLET MICROFLUIDICS

(71) Applicant: City University of Hong Kong, Hong Kong (HK)

(72) Inventors: Dong SUN, Hong Kong (HK); Jiayu SUN, Hong Kong (HK)

(21) Appl. No.: 17/841,695

(22) Filed: Jun. 16, 2022

(51) Int. Cl. C12N 15/87; C12N 5/00; C12N 5/077; B01L 3/00

(52) U.S. Cl. CPC C12N 15/87; C12N 5/0012; C12N 5/0658; B01L 3/502761; B01L 2300/0867; B01L 2300/0654; B01L 2200/0647; B01L 2200/0673; B01L 2400/086; C12N 2510/00; G01N 21/6458

(57) ABSTRACT

A system for quantitative control of mitochondrial transfer based on droplet microfluidics includes a droplet generation module configured to generate droplets containing isolated mitochondria and a single cell; a droplet observation module configured for observation of the generated droplets under a microscope; and a droplet collection module configured to collect the generated droplets. The number of mitochondria needed to be transferred into recipient cells is an import issue in precise medicine. The development of the presented invention, which can achieve a precise quantity-control on mitochondrial transfer at the single cell level, can help us to determine the mitochondria number needed to make a significant function improvement on the recipient cells before conducting the cell therapy for mtDNA-related diseases.

### FIELD OF THE INVENTION

The present invention relates to mitochondrial transfer based on a droplet microfluidics system, and more particularly, to high-efficiency quantitative mitochondrial transfer based on a droplet microfluidics system.

### BACKGROUND

Heteroplasmy, which is the ratio of mutant to wild-type mitochondria DNA (mtDNA), determines the severity of mitochondria-related disorders. In muscle tissues, when heteroplasmy exceeds a certain level or the mitochondria becomes dysfunctional, less ATP and excessive levels of reactive oxygen species (ROS) are produced, which can trigger muscle atrophy, weakness, and loss of endurance. Previous clinical and preclinical animal studies demonstrated links between increased mitochondrial damage and poor skeletal muscle health. Since 1990s, cell therapy, especially myoblast transplantation, has been proposed to improve the regeneration of injured skeletal muscles. However, failure of early clinical trials with myoblast transplantation has been observed due to host immune cells such as CD8+ T lymphocytes causing massive cell death. The accumulation of immune cells produces sarcolemma damage, induces activation of caspase 3 in myofibers, and further induces apoptosis in muscle fibers. Therefore, new approaches, such as macrophages regulation and chemical induction of stem cells, need to be developed to treat skeletal muscle disorders. Restoring or improving mitochondrial functions to facilitate muscle regeneration is an attractive approach.

Apart from energy production for cells, the mitochondria can influence cell proliferation, aging, apoptosis, innate immunity, calcium homeostasis, and even stem cell differentiation potential. Mutations of mtDNA impair the functions of cells and tissues. It is observed that spontaneous transfer of mitochondria can occur in nature between healthy and damaged cells via different mechanisms, which is believed to protect the damaged cells and restore their cellular function. Mitochondrial transfer is a technique to alter mtDNA in cells, and has attracted more and more attention since it was first reported by Clark and Shay. Mitochondrial transfer has already been used in cell therapy for mtDNA-related diseases. It is easier to perform and practically more efficient than other techniques modifying the mitochondrial genome, such as mitoZFNs and mitoTALENs. Transferring exogenous mitochondria into recipient cells could also reduce the ratio of mutant to wild-type mtDNA, and promote the restoration or improvement of cell and tissue functions. Previous studies showed that exogenous isolated mitochondria could be delivered into cells via co-culture or microinjection. In the method of co-culture with isolated mitochondria, the isolated mitochondria are engulfed by the recipient cells via endocytosis, which is a kind of cellular activity to uptake objects ranging from nanometers to several micrometers from the surrounding environment. The isolated mitochondria randomly move around the recipient cell and have chances to be engulfed by the cell when contacting it; this phenomenon could be regarded as a random and sporadic process. The transfer efficiency of co-culture method is influenced by the quantity of extracellular isolated mitochondria. Though it could reach the highest at 28% in a previous study, the number of transferred mitochondria is considerably heterogeneous (1-60 mitochondria per recipient cells) even when subjected to an equal amount of isolated mitochondria. Despite being a rather simple process, the success of the co-culture method is dependent on many uncontrollable factors that underlie the unsatisfactory cell metabolism recovery rate of recipient cells in previous work (˜0.2%). An automated optical tweezers-based manipulation system (OT-based manipulation system) was used for qualitative and quantitative mitochondrial transfer to reduce influences from uncontrollable factors. The OT-based system could precisely pick up the healthy mitochondria and transport them to the target recipient cell. However, this method suffers from the limitation of low throughput, which makes it difficult to be used for clinical applications.

Unlike co-culture methods, microinjection injects the isolated mitochondria preloaded in a microneedle directly into the recipient cells; thus, it may cause damage to the recipient cells owing to the cell membrane opening during the delivery process. In addition, the throughput of the microinjection technique is low.

All the methods mentioned above provide useful tools for studying the mechanism of cell functions' restoration or improvement upon mitochondrial transfer. However, they still could not fulfill the demand of a large quantity of mitochondria-transferred recipient cells in the cell therapy industry. The co-culture technique shows considerable advantage due to being harmless, but its low efficiency and heterogeneity are still a major bottleneck.

Droplet microfluidics is a technology that disperses continuous flow carrying chemical reagents, cells, or other biomaterials into discrete volumes at micrometer scale, called droplets. These droplets are the basic unit for further chemical reactions, cell life activities, target detections, and material synthesis. Droplet microfluidics provides a much smaller and constrained environment than the bulk volume method, thus allowing more rapid reaction and detection of molecules/particles and interactions with encapsulated cells. Previous works have demonstrated that the droplet generation rates could be as high as thousands of droplets per second, making droplet microfluidics a high-throughput technique intrinsically. An important application of droplet microfluidics is single-cell analysis, in which a single cell is encapsulated in one droplet for analyzing cell life activities or cell modification, such as antibody analysis or gene editing.

U.S. Patent Application Publication No. 2017/159017A1 entitled: “Method for introducing exogenous mitochondria into a mammalian cell” and US patent U.S. Patent Application Publication No. 2013/149778A1 entitled: “Method and Applications of Peptide-Mediated Mitochondrial Delivery System” applies natural cell membrane engulfing processes also known as endocytosis to transfer isolated mitochondria.

U.S. Patent Application Publication No. 2019/276852A1 entitled: “Method for delivering exogenous mitochondria into cells” and Europe Patent Application Publication No. EP3169338A1 entitled: “Methods for the intercellular transfer of isolated mitochondria in recipient cells” apply the centrifugation method to transfer mitochondria. In these methods, isolated mitochondria and cells were centrifuged together, which improved the mitochondria transfer efficiency by forcing mitochondria into the cells.

U.S. Pat. No. 10,760,040B1 entitled: “Mechanical transfection devices and methods” applies mechanical forces induced by the fluid to open the cell membrane and deliver isolated mitochondria into the cells.

The traditional microinjection method causes physical harm to recipient cells. The co-culture technology is able to generate the number of cells required for cell therapy. However, the co-culture technology cannot control the number of mitochondria transferred into each recipient cell, so some cells that do not have enough mitochondria transferred may not be able to perform their full therapeutic function.

### SUMMARY OF THE INVENTION

Therefore, a mitochondrial transfer based on droplet microfluidics system is provided as a high-efficiency quantitative mitochondrial transfer system. In the present invention, droplet microfluidics-based mitochondrial transfer method causes only slight/minor physical harm to recipient cells, which is unavoidable in microinjection, and shows high-throughput advantage. Compared with the traditional co-culture method, the proposed method could also control the number of mitochondria transferred to the recipient cells at the single-cell level, and achieve high efficiency and high throughput.

The present invention provides a high-efficiency quantitative mitochondrial transfer based on a droplet microfluidics system. A droplet generation module configured to generate droplets containing isolated mitochondria and a single cell; a droplet observation module configured for observation of the generated droplets under a microscope; and a droplet collection module configured to collect the generated droplets.

In another embodiment, the droplet generation and observation modules are connected by a conduit.

In other embodiments, the system is disposed on a chip.

In yet another embodiment, the chip is smaller than 8 cm in length.

In a further embodiment, the droplet generation module comprises three inlets.

In another embodiment, the droplet generation module further comprises mitochondrial recipient cell suspension, isolated mitochondria suspension, and surfactant-added fluorinated oil.

In other embodiments, the mitochondrial recipient cell is recipient C2C12 cell.

In a further embodiment, the droplet generation module comprises a flow-focusing structure configured to separate the mitochondrial recipient cell suspension and the isolated mitochondria suspension into droplets.

In yet another embodiment, the droplet generation module comprises a wave-like structure which is configured to focus randomly distributed cells from the inlet to a line.

In other embodiment, the wave-like structure is configured to improve the single cell encapsulation ratio more than 47%.

In a further embodiment, the wave-like structure is configured to suppress the multiple cell encapsulation ratio less than 6%. In other embodiments, the droplet comprises isolated mitochondria and a single

cell has a diameter of 40 μm.

In another embodiment, an efficiency of mitochondrial transfer is at least 75%.

In other embodiments, the system is configured to yield at least 2×10⁶ recipient cells in the droplets for mitochondria transfer in 30 minutes.

The present invention also provides a method for quantitative control of mitochondrial transfer based on droplet microfluidics, comprising the steps of preparing a first suspension, a second suspension, and an oil fluid; co-flowing of the first suspension, the second suspension, and the oil fluid to a system for quantitative control of mitochondrial transfer based on droplet microfluidics; co-encapsulating the first and the second suspensions in droplets; collecting the droplets; and co-culturing the first and the second suspensions in droplets.

In a further embodiment, the first suspension is a mitochondrial recipient cell suspension and the isolated mitochondria suspension.

In yet another embodiment, the second suspension is an isolated mitochondria suspension.

In other embodiments, the droplets have a diameter of 40 μm.

In other embodiments, the system comprises a wave-like structure.

In a further embodiment, the droplets comprise mitochondria, mitochondrial recipient cell, and mitochondrial recipient cell.

### BRIEF DESCRIPTION OF THE DRAWINGS

FIGS. 1A-1G show the schematic of the system setup, workflow of the droplet microfluidics-based mitochondrial transfer technique and experimental assessments. FIG. 1A shows a system setup for the droplet microfluidics-based mitochondria transfer technique. FIG. 1B shows a wave-like structure for cell pairing before encapsulation and wave-like structure for mitochondria and cell suspension mixing after encapsulation. FIG. 1C shows a co-encapsulation of cells and mitochondria in droplets. FIG. 1D shows a demonstration of cell focusing on the wave-like structure. FIG. 1E shows a demonstration of mitochondrial transfer process via endocytosis in the droplet. FIG. 1F shows fabricated chip for droplet generation and observation. FIG. 1G shows an experimental workflow to demonstrate the effectiveness of mitochondria-transferred recipient cells on myogenic differentiation in-vitro.

FIGS. 2A-2I depict a presentation of droplet microfluidics-based mitochondrial transfer system. FIG. 2A shows a co-flowing of cell suspension and isolated mitochondrial suspension to the wave-like structure. FIG. 2B shows a wave-like structure for cells focusing on the increase in single-cell encapsulation efficiency. FIG. 2C shows a flow-focusing structure for droplet generation. FIG. 2D shows an isolated mitochondria (stained with MitoTracker™ Green FM before isolation) from donor cells (C2C12 myoblasts). FIG. 2E shows an isolated mitochondria taken up by the recipient C2C12 cell, and a portion of droplets collected for further confocal imaging analysis to confirm the success of mitochondrial transfer (recipient cell in red and transferred mitochondria in green). FIG. 2F shows a cell encapsulation efficiency using the wave-like structure under different concentrations of cell suspension. Here 1 and 2 stand for one and two or more cells encapsulated in one droplet, respectively; L stands for 0.85×10⁷ cell/mL; and H stands for 1.7×10⁷ cell/mL. FIG. 2G shows the influence of Oil/Water flow rate ratio on droplet size. FIG. 2H shows the influence of droplet size on mitochondrial transfer efficiency. FIG. 2I shows the influence of cell suspension flow rate on cell viability. All data from FIGS. 2F-2I were presented as mean±SD and analyzed by one-way ANOVA with Dunn's multiple comparisons test. *p<0.05 and **p<0.01. The red arrows from A to C indicated the flow directions in the channel of the microfluidic chip.

FIGS. 3A-3B depict a number of isolated mitochondria encapsulated in each droplet at different concentrations of isolated mitochondrial suspension used. FIG. 3A shows the 3D rebuilding images of isolated mitochondria (labelled with MitoTracker™ Green) encapsulated in one droplet at three different concentrations of isolated mitochondrial suspension (cells stained with Cell Mask Deep Red were not shown here by closing the red color channel of confocal microscope). Every frame here contained one droplet, and the number of isolated mitochondria was labeled on the top left corner. FIG. 3B shows the number of isolated mitochondria encapsulated in each droplet at three different concentrations of isolated mitochondria suspension. All data were presented as mean±SD.

FIGS. 4A-4C depict a quantitative control of mitochondrial transfer using the proposed droplet-based method. FIG. 4A shows the representative confocal images of recipient cell suspension after mitochondrial transfer at 0.25, 0.5, or 1.0 units, separately. One unit of mitochondria stands for concentration of mitochondria isolated from 1×10⁶ cells and suspended in 10 μL mitochondria storing reagent. The transferred mitochondria were labelled with MitoTracker™ Green FM before isolation, and the recipient cells were labelled with Cell Mask Deep Red before encapsulation into droplets. Three recipient cells from each unit were selected to illustrate the localization of the transferred mitochondria inside the cells (bottom). FIG. 4B shows the average number of transferred mitochondria per cells under different mitochondria concentrations. Mitochondria numbers were counted within the 3D rebuilding images under the confocal fluorescence microscope. FIG. 4C shows the transfer efficiency calculated as the ratio of the number of isolated mitochondria transferred into a recipient cell to the total number of isolated mitochondria encapsulated in a droplet. All data in FIG. 4B and FIG. 4C were presented as mean±SD.

FIGS. 5A-5D depict an in-vitro study of mitochondrial transfer on myogenic differentiation of C2C12 myoblast cells. FIG. 5A shows the representative brightfield images of mitochondrial transferred C2C12 cells during myogenic induction process. C2C12 cells were subjected to mitochondrial transfer at different concentrations before myogenic induction (8, 14, and 31 exogenous isolated mitochondria transferred per cell were defined as low-mito, middle-mito and high-mito transferred group, respectively). Cell morphology and formed myotubes were imaged right before induction started and on days 3 and 7 of induction process. FIGS. 5B-5C shows on day 7, myotube area and length were determined by ImageJ, and three FOVs were taken per well. FIG. 5D shows the cellular proliferation of C2C12 cells was determined by MTT assay on days 1, 2, 3, and 4 post mitochondrial transfer. All the values were normalized to day 0. Data were presented as mean±SD and analyzed with one-way ANOVA followed by Dunn's multiple comparison test, N=3. * (or #) p<0.05, ** (or ##) p<0.01, *** (or ###) p<0.001, and **** (or ####) p<0.0001.

### DETAILED DESCRIPTION

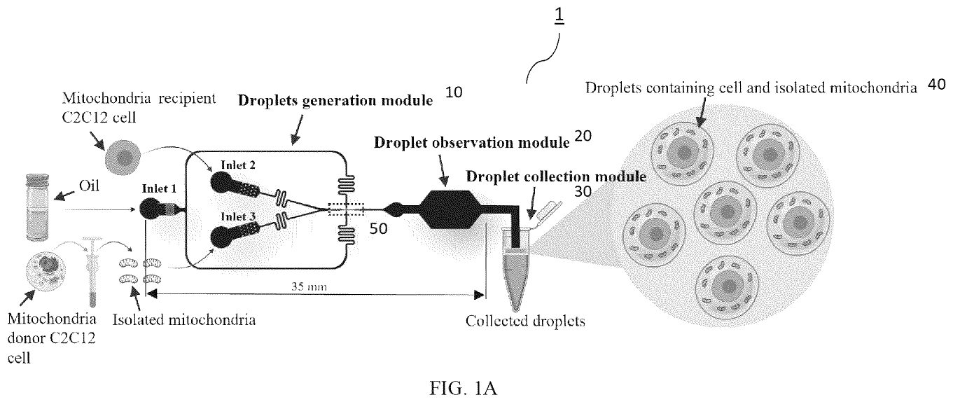

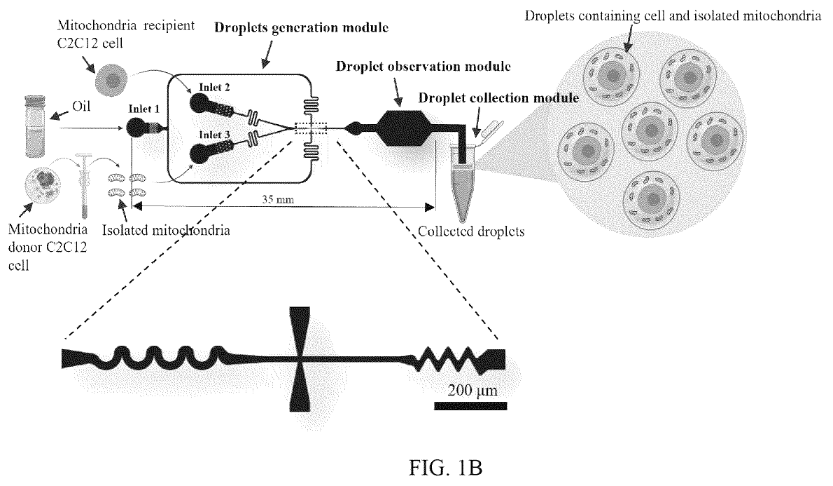

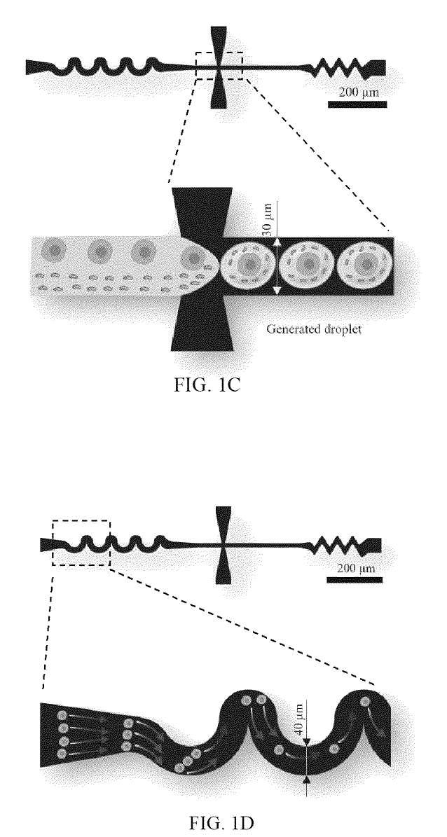

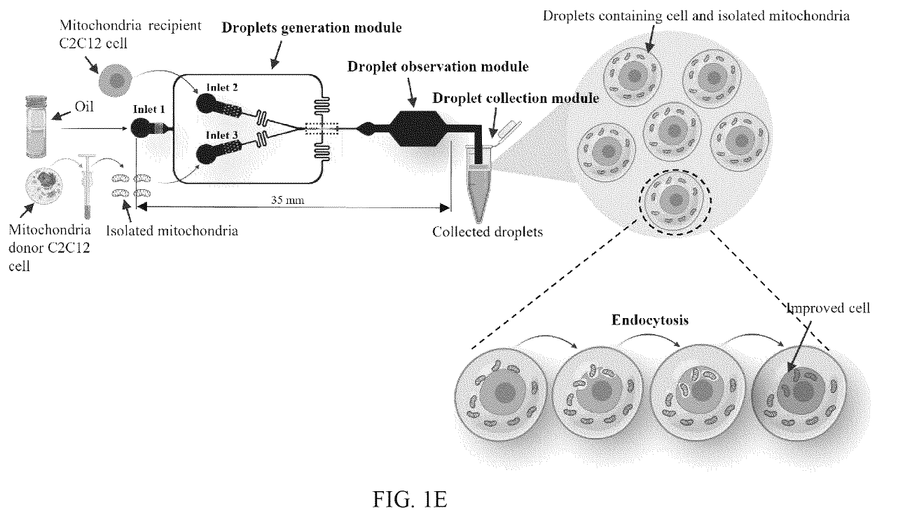

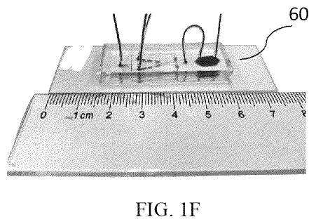

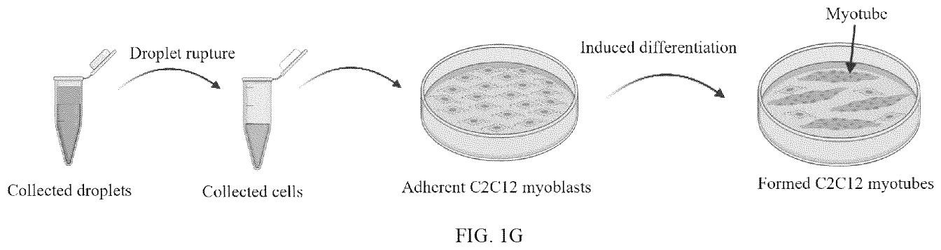

Turning to the drawings in detail, FIG. 1A schematically depicts a high-efficiency quantitative mitochondrial transfer based on a droplet microfluidics system. A droplet generation module 10 is configured to generate droplets 40 containing isolated mitochondria and a single cell. The droplet generation module 10 also comprises a wave-like structure which is configured to focus randomly distributed cells from the inlet to a line to improve the single cell encapsulation ratio and suppress the multiple cell encapsulation ratio. Element 20 is a droplet observation module configured for observation of the generated droplets under a microscope (not shown). Element 30 is a droplet collection module configured to collect the generated droplets 40. The droplet generation and observation modules may be connected by a conduit 50. Optionally, the system 1 is disposed on a chip 60 as seen in FIG. 1F.

### Microfluidic Chip Fabrication and Operations

The designed chip was fabricated using soft-lithography. Prior to experiments, the chip channels were coated with a surface modification agent to make them hydrophobic for stable water-in-oil droplet generation and transporting.

### Cell Culture

C2C12 myoblasts were cultured in Dulbecco modified Eagle medium (DMEM) with high glucose (Gibco™, 11965084) supplemented with 10% fetal bovine serum (Gibco™, 12800058) and 1% Antibiotic-Antimycotic (Gibco™, 15240096) at 37° C. in 5% CO₂

### Mitochondria Isolation

The mitochondria used in this work were freshly isolated from C2C12 myoblasts cells by following the protocol of mitochondria isolation kit (Beyotime, C3601) before each mitochondrial transfer experiment. First, the mitochondria of donor C2C12 cells were stained with MitoTracker™ Green FM (Invitrogen™, M7514). Second, the stained cells were washed three times with PBS, detached from the culture flask with Trypsin/EDTA Solution (Gibco™, R001100), and centrifuged at 500 g for 5 minutes. Third, the supernatant was removed, and the collected cells were resuspended with 1 ml of cell lysis reagent (Beyotime, C3601-1) and placed in ice bath for 15 minutes. Fourth, the lysed cells were homogenized with a glass homogenizer for 30 cycles. Fifth, the homogenized cells were centrifuged at 1,000 g for 10 minutes at 4° C. Then, the supernatant was resuspended with 1 ml of cell lysis reagent (Beyotime, C3601-1) and centrifuged at 1,000 g for 10 minutes at 4° C. again for more purity. Finally, the supernatant was collected and centrifuged at 3,500 g for 10 minutes at 4° C. The pellet collected was the isolated mitochondria. Mitochondria storage reagent (Beyotime, C3601-3) was used to suspend the isolated mitochondria at the required concentration for further experiments. The mitochondria isolated from 1×10⁶ cells and suspended in 10 μL of mitochondria storing reagent was set as one unit of mitochondrial suspension.

### 3D Reconstruction of Cells and Mitochondria Under Confocal Fluorescence Microscope

After co-culturing the recipient cells and exogenous isolated mitochondria in droplets for 2 hours, the droplets were loaded into the observation module, as shown in FIG. 1A. Then, the confocal fluorescence microscope (LEICA SP8LIA++ TRUE Confocal Laser Scanning Microscope) was used to take a slice of droplets containing single cells (labelled with Cell Mask Deep Red) and isolated mitochondria (labelled with MitoTracker™ Green FM). Afterwards, 3D reconstruction of the taken slices was conducted using the confocal microscopy software. Finally, the isolated mitochondria transferred into the cell were counted as the green particles inside the red cell region, and the green particles outside the red cell region were counted as the un-transferred isolated mitochondria. Mitochondrial transfer efficiency was calculated as the ratio of the transferred mitochondria to the total mitochondria inside the droplet. The same process was repeated for cells recovered from droplets to count the number of mitochondria transferred under different concentration of isolated mitochondria concentration used.

### Droplet Rupture and Cell Collection

The collected droplets floated on the top the fluorinated oil. Before the droplets broke, the extra oil on the bottom of the tube was removed. Then, 1 ml of 50% 1H,1H,2H,2H-Perfluorooctanol (PFO, Thermo Scientific™, AAB2015609) was added to the tube containing the collected droplets and gently vortexed for 1 minute for the droplets to merge into a bulk solution. Afterwards, the upper bulk solution was carefully moved to a new tube and centrifuged at 300 g for 3 minutes to collect the cells.

### Myogenic Differentiation of C2C12

For evaluating C2C12 differentiation, 5,000 cell/cm² were seeded in a six-well plate and cultured in growth media until reaching 80% confluence. The media were then replaced with DMEM (Gibco™, 11965084) supplemented with 2% horse serum (Gibco™, 16050130). The cells were kept in differentiation medium until the end of the assay, typically between day 5 to day 7. Myotube formation was monitored every two days. The time-points were days 0, 3, and 7.

### MTT Assay

The cell proliferation rate of each C2C12 cell group was determined by MTT assay. In brief, C2C12 cells were plated at a density of 5,000 cell/cm² in 96-well plates and incubated for 24 h. After incubation, the cells were treated with 3-(4,5-dimethyl-2-thiazolyl)-2,5-diphenyl-2H-tetrazolium bromide (MTT, 100 μL, 0.5 mg/mL) for 3 hours at 37° C. The produced dark blue formazan crystals were solubilized by 100 μL DMSO. The absorbance at 570 nm was measured with a microplate reader.

In use, the developed droplet-based mitochondria transfer system of FIG. 1A employs. Three kinds of solutions such as (1) mitochondrial recipient C2C12 cell suspension, (2) isolated mitochondria suspension, and (3) surfactant-added fluorinated oil. Then, mitochondrial recipient C2C12 cell suspension, isolated mitochondria suspension, and surfactant-added fluorinated oil (Sphere Fluidics, C021) are loaded into the droplet generation module 10 via three inlets. After that, the generated droplets are collected in the droplet collection module 30. Where the droplets include mitochondria, mitochondrial recipient cell, and mitochondrial recipient cell with mitochondria inside.

As shown in FIG. 1B and 1C, the flow-focusing structure is used to separate the two suspensions into droplets. The two suspensions are mitochondrial recipient C2C12 cell suspension and isolated mitochondria suspension. A wave-like structure is adapted to focus the randomly distributed cells from the inlet to a line as shown FIG. 1D. Thus, the single-cell encapsulation efficiency can be improved. The isolated mitochondria are taken up by the recipient C2C12 cells via endocytosis inside the droplets, as indicated in FIG. 1E. The droplet generation and observation modules are connected via a PE tubing (BD Intramedic™, BD 427406), and the whole fabricated chip is only approximately 8 cm in length as shown in FIG. 1F. After mitochondrial transfer is completed, the recipient cells are collected from the droplets via a droplet rupture process, and functional experiments could be immediately performed to assess the effect of mitochondrial transfer at different numbers on C2C12 cell myogenic differentiation in-vitro, as indicated in FIG. 1G.

For the observation and 3D rebuilding under a confocal fluorescence microscope (LEICA SP8LIA++ TRUE Confocal Laser Scanning Microscope), MitoTracker™ Green FM (Invitrogen™, M7514) at a concentration of 2 μM is used to stain the mitochondria before isolation from donor cells.

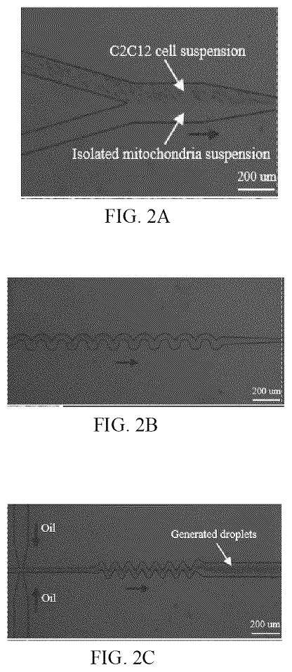

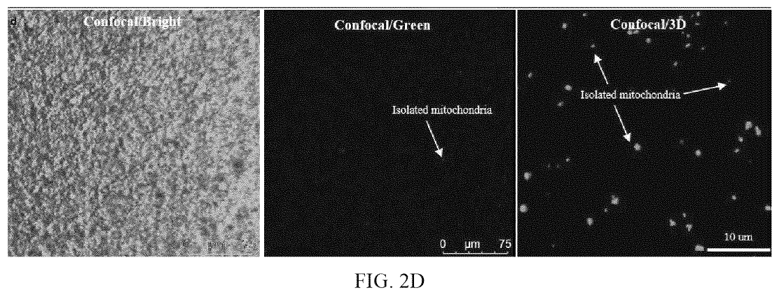

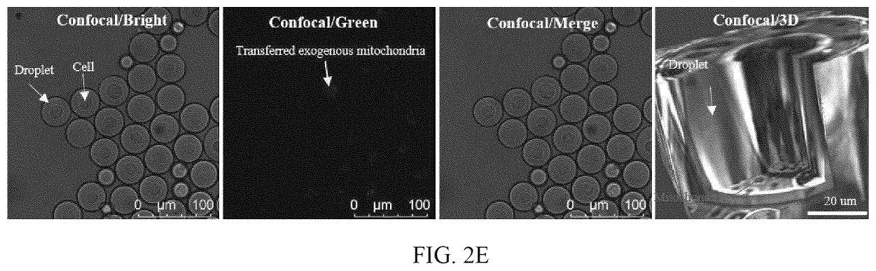

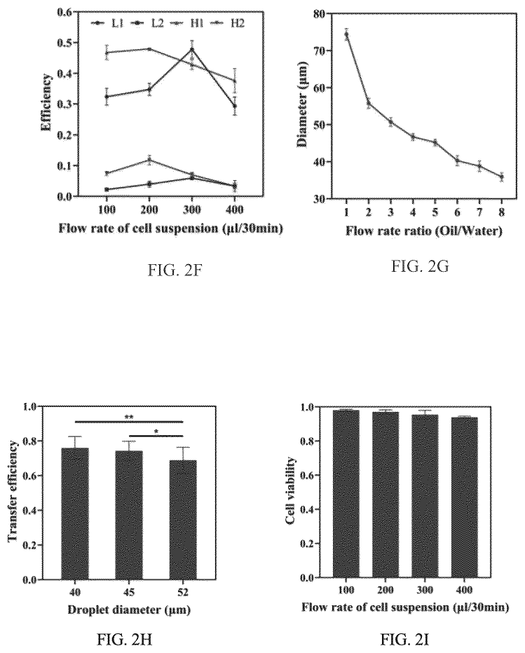

As presented in FIGS. 2A-2C, after mitochondria isolation, the mitochondria recipient cells and the freshly isolated mitochondria are loaded into the microfluidic chip immediately for the encapsulation and mitochondrial transfer process. The labelled isolated mitochondria in suspension are spherical-like structures with a diameter of around 1 μm as shown in FIG. 2D. After 2 hours of co-culture in the generated droplets, FIG. 2E shows that the isolated mitochondria are taken up by the recipient cells via endocytosis.

As mentioned above, the cell encapsulation efficiency is improved beyond the Poisson distribution by using the wave-like structure. FIG. 2F shows that the single-cell encapsulation efficiency is able to reach approximately 47.8%, while the multiple-cell encapsulation ratio is suppressed to approximately 5.9% at a situation while using cell concentration of 0.85×10⁷ cell/mL and a cell suspension flow rate of 300 μL/30 mins (the flow rate of isolated mitochondria suspension was maintained to be the same). This implies that the single-to-multiple cell encapsulation ratio is increased to 8.1. In other words, 292% of the Poisson distribution (at the condition of cells encapsulated per droplet was 0.6). Such improvement in single-cell encapsulation efficiency is able to help increase the throughput. The proposed system is able to notably yield 2×10⁶ recipient cells in droplets for mitochondria transfer in 30 minutes. As shown in FIG. 2H, the mitochondrial transfer efficiency, defined as the ratio of the isolated mitochondria transferred into the cell to the total isolated mitochondria encapsulated in the droplet, decreased slightly from 75% to 70% with the increase in droplet diameter from 40 μm to 52 μm. As shown in FIG. 2G, the droplet diameter is set to 40 μm by setting the flow rate ratio of oil/water phase to 6. As shown in FIG. 2I, after processing with the developed system, the viability of the recipient cells can still be kept relatively high, such as 95% at a flow rate of 300 μL/30 mins.

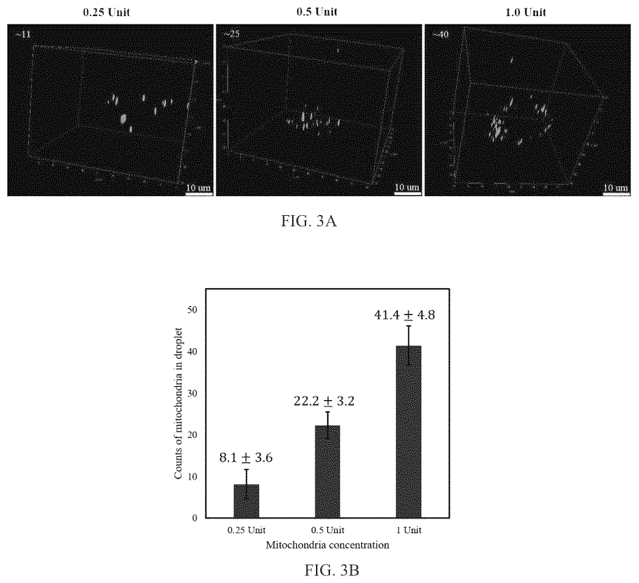

The closed microenvironment of droplets limits the travelling distance of isolated mitochondria and increases the probability of the isolated mitochondria to contact with the cell, thereby making mitochondria taken up by the cell easily, and improving the mitochondria transfer efficiency. Moreover, due to the smaller size of isolated mitochondria than that of the droplets (1-40 μm in diameter), the isolated mitochondria are evenly encapsulated in each droplet. The number of isolated mitochondria encapsulated in droplets can be controlled by adjusting the concentration of isolated mitochondria suspension. FIGS. 3A-3B show that 8, 22, and 41 isolated mitochondria are encapsulated in each droplet at 0.25, 0.5 and 1.0 unit concentrations of isolated mitochondria suspension used, respectively. The 1.0 unit of concentration refers to mitochondria isolated from 1×10⁶ cells and suspended in 10 μL mitochondrial storing reagent (Beyotime, C3601-3).

### EXAMPLE

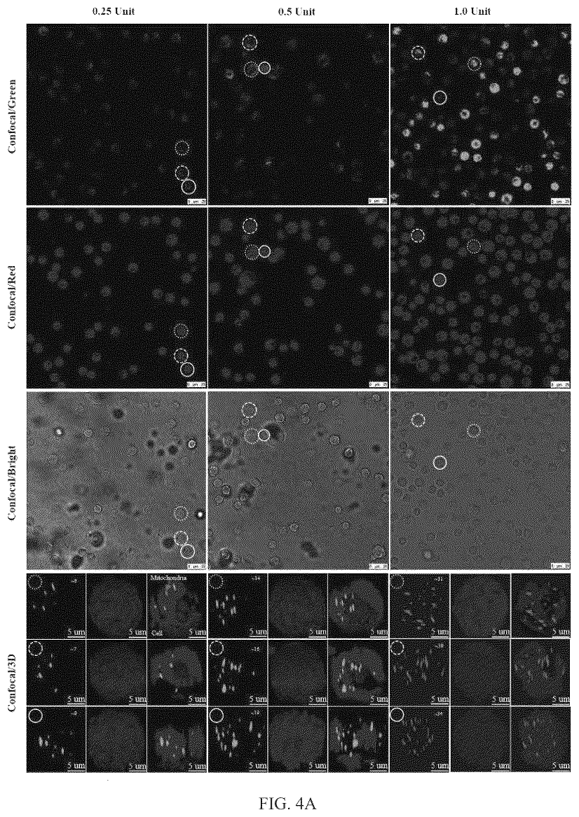

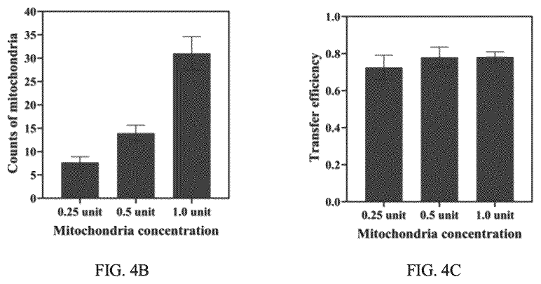

In one embodiment, three different concentrations of isolated mitochondrial suspension (0.25, 0.5, and 1.0 units of concentration) are used to verify the transfer efficiency of the present invention. FIGS. 4A and 4B show that under 0.25, 0.5, and 1.0 units of concentration used, 8, 14, and 31 isolated mitochondria are transferred into the recipient cells on average, respectively. FIG. 4C shows that under 0.25, 0.5, and 1.0 units of concentration used, the mitochondrial transfer efficiency is approximately 75%.

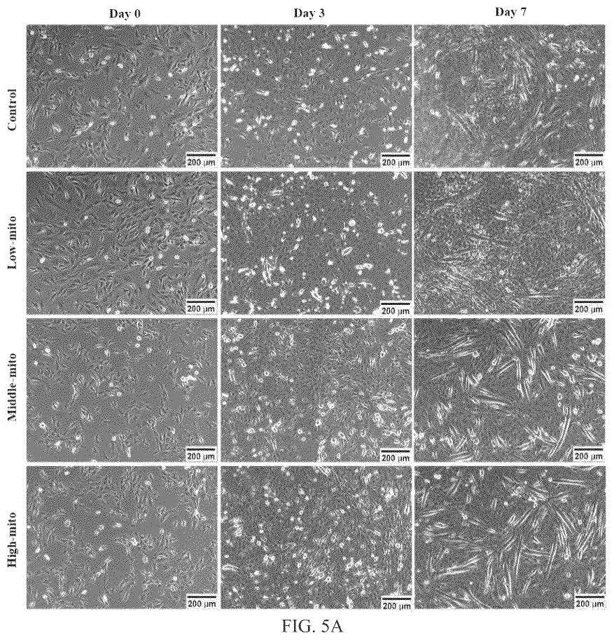

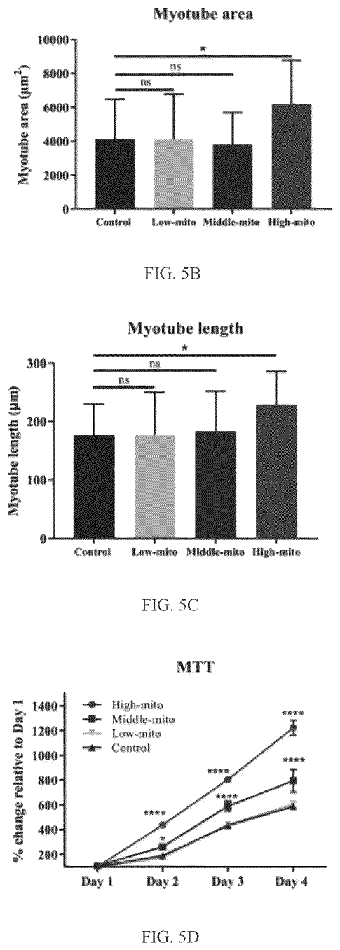

Myogenesis assay is performed to test the differentiation ability of C2C12 myoblasts after mitochondrial transfer with the proposed droplet microfluidics-based method. After 7 days of induction, FIGS. 5A-5C show that the myotube area and length (as indicators of myogenesis) are significantly increased in the high-mito transferred group (31 exogenous isolated mitochondria transferred per cell) compared with the control, low-mito, and mid-mito transferred groups (corresponding to 0, 8 and 14 exogenous isolated mitochondria transferred per cell, respectively). Moreover, MTT assay indicates 2.5 and 1.5 times increase in cell proliferation on day 4 in the high and mid-mito transferred groups, respectively, as shown in FID. 5D.

### Industrial Applicability

The present invention provides a mitochondrial transfer based on droplet microfluidics system is provided as a high-efficiency quantitative mitochondrial transfer system due to the following advantages:

The number of mitochondria needed to be transferred into recipient cells is an import issue in precise medicine. The development of the presented invention, which can achieve a precise quantity-control on mitochondrial transfer at the single cell level, can help us to determine the mitochondria number needed to make a significant function improvement on the recipient cells before conducting the cell therapy for mtDNA-related diseases.

Compared to existing mitochondrial transfer methods, the invented method can produce massive quantitative mitochondria transferred cells for cell therapy purpose. Although the co-culture with isolated mitochondria method can also produce the number of cells needed for cell therapy, it cannot control the number of mitochondria transferred into each recipient cell, thus, some cells without enough mitochondria transferred may cannot fully play their therapeutic functions. However, using the invented system, the mitochondria transferred into each recipient cell is controlled, thus, the cells used for cell therapy can fully play their therapeutic functions.

While the present disclosure has been described and illustrated with reference to specific embodiments thereof, these descriptions and illustrations are not limiting. It should be understood by those skilled in the art that various changes may be made and equivalents may be substituted without departing from the true spirit and scope of the present disclosure as defined by the appended claims. The illustrations may not necessarily be drawn to scale. There may be distinctions between the artistic renditions in the present disclosure and the actual apparatus due to manufacturing processes and tolerances. There may be other embodiments of the present disclosure which are not specifically illustrated. The specification and the drawings are to be regarded as illustrative rather than restrictive. Modifications may be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations.

What is claimed is:

1. A system for quantitative control of mitochondrial transfer based on droplet microfluidics, comprising:
a droplet generation module configured to generate droplets containing isolated mitochondria and a single cell;
a droplet observation module configured for observation of the generated droplets under a microscope; and
a droplet collection module configured to collect the generated droplets.

2. The system according to claim 1, wherein the droplet generation and observation modules are connected by a conduit.

3. The system according to claim 1, wherein the system is disposed on a chip.

4. The system according to claim 4, wherein the chip is smaller than 8 cm in length.

5. The system according to claim 1, wherein the droplet generation module comprises three inlets.

6. The system according to claim 1, wherein the droplet generation module further comprises mitochondrial recipient cell suspension, isolated mitochondria suspension, and surfactant-added fluorinated oil.

7. The system according to claim 6, wherein the mitochondrial recipient cell is recipient C2C12 cell.

8. The system according to claim 1, wherein the droplet generation module comprises a flow-focusing structure configured to separate the mitochondrial recipient cell suspension and the isolated mitochondria suspension into droplets.

9. The system according to claim 8, wherein the droplet generation module comprises a wave-like structure which is configured to focus randomly distributed cells from the inlet to a line. The system according to claim 9, wherein the wave-like structure is configured to improve the single cell encapsulation ratio more than 47%.

11. The system according to claim 9, wherein the wave-like structure is configured to suppress the multiple cell encapsulation ratio less than 6%.

12. The system according to claim 1, wherein the droplet comprises isolated mitochondria and a single cell has a diameter of at less than 40 μm.

13. The system according to claim 1, wherein an efficiency of mitochondrial transfer is at least 75%.

14. The system according to claim 1, wherein the system is configured to yield at least 2×10⁶ recipient cells in the droplets for mitochondria transfer in 30 minutes.

15. A method for quantitative control of mitochondrial transfer based on droplet microfluidics, comprising the steps of:
preparing a first suspension, a second suspension, and an oil fluid;
co-flowing of the first suspension, the second suspension, and the oil fluid to a system for quantitative control of mitochondrial transfer based on droplet microfluidics;
co-encapsulating the first and the second suspensions in droplets;
collecting the droplets; and
co-culturing the first and the second suspensions in droplets.

16. The method according to claim 15, wherein the first suspension is a mitochondrial recipient cell suspension and the isolated mitochondria suspension.

17. The method according to claim 15, wherein the second suspension is an isolated mitochondria suspension.

18. The method according to claim 15, wherein the droplets have a diameter of at less than 40 μm.

19. The method according to claim 15, wherein the system comprises a wave-like structure.

20. The method according to claim 15, wherein the wherein the droplets comprise mitochondria, mitochondrial recipient cell, and mitochondrial recipient cell with mitochondria inside.
